(19) United States
(12) Patent Application Publication
(10) Pub. No.: US 2023/0063813 A1
(43) Pub. Date: Mar. 2, 2023

(54) Ambulatory Blood Pressure Cuff

(71) Applicant: Apple Inc., Cupertino, CA (US)

(72) Inventors: William K. Smith, San Francisco, CA (US); Motohide Hatanaka, Menlo Park, CA (US); Nicholas R. Trincia, San Francisco, CA (US); Wegene H. Tadele, San Francisco, CA (US); Albert Wang, Sunnyvale, CA (US); Salome Bavetta, San Jose, CA (US)

(21) Appl. No.: 17/899,443

(22) Filed: Aug. 30, 2022

Related U.S. Application Data
(60) Provisional application No. 63/238,701, filed on Aug. 30, 2021.

(51) Int. Cl. A61B 5/022; A61B 5/0225; A61B 5/00

(52) U.S. Cl. CPC A61B 5/02233; A61B 5/0225; A61B 5/6824; A61B 5/6831

(57) ABSTRACT

Embodiments of this disclosure are directed to a wearable blood pressure measurement device having an inner sleeve and a cuff module. The inner sleeve is wearable on a limb of a user and has a first attachment mechanism. The cuff module has a second attachment mechanism that is attachable to the first attachment mechanism. The cuff module includes a cuff configured for placement over the inner sleeve, a bladder assembly having one or more bladders configured to expand and contract in response to a flow of a fluid therethrough, and a controller. The controller is configured to measure a blood pressure of the user while controlling expansion and contraction of the one or more bladders.

### CROSS-REFERENCE TO RELATED APPLICATION

This application is a nonprovisional of, and claims the benefit under 35 U.S.C. § 119(e) of, and priority to U.S. Provisional Patent Application No. 63/238,701, filed Aug. 30, 2021, the contents of which are incorporated herein by reference in its entirety.

### FIELD

The described embodiments relate generally to wearable devices. More particularly, the described embodiments relate to ambulatory blood pressure cuffs that can be worn around a limb of a user to, periodically or continuously, measure a blood pressure of the user during daily activities of the user.

### BACKGROUND

A user may measure blood pressure by attaching a blood pressure measurement device to one of their limbs. The blood pressure measurement device may include an inextensible cuff that secures an inflatable bladder against a limb of the user. The inflatable bladder can be expanded, and the inextensible cuff may cause the bladder to compress the limb, thereby compressing one or more blood vessels in the limb and restricting and/or stopping (i.e., occluding) blood flow through the vessels. The various pressures in the inflatable bladder that restrict and/or stop blood flow through the vessels in the limb may be measured and used to determine the blood pressure of the user.

A blood pressure measurement device including a blood pressure cuff is typically worn during measurement of a blood pressure, and is removed promptly thereafter. In some cases, it might be desirable to wear the monitoring device for longer periods of time such that physiological measurements can be performed periodically or continuously.

### SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

Embodiments of this disclosure are directed to a wearable blood pressure measurement device having an inner sleeve and a cuff module. The inner sleeve may be wearable on a limb of a user and having a first attachment mechanism. The cuff module may have a second attachment mechanism that is attachable to the first attachment mechanism. The cuff module may include a cuff configured for placement over the inner sleeve, a bladder assembly having one or more bladders configured to expand and contract in response to a flow of a fluid (e.g., one or more gases (e.g., air) or liquids (e.g., oil)) therethrough, and a controller. The controller may be configured to measure a blood pressure of the user while controlling expansion and contraction of the one or more bladders.

Embodiments of this disclosure are also directed to a wearable device having an inner sleeve and a measurement module. The inner sleeve may be wearable on a limb of a user. The measurement module may be removably coupled to the inner sleeve. The measurement module may include one or more sensors, a controller, and a power delivery system. The controller may be in communication with the one or more sensors and configured to measure one or more health parameters of the user using the one or more sensors. The power delivery system may be configured to supply power to the one or more sensors and the controllers.

Embodiments of this disclosure are also directed to a wearable device for attachment to a limb of a user. The wearable device may include an elongate strap having a first end and a second end, a fold between the first end and the second end in the elongate strap, and a sizing mechanism. The sizing mechanism may allow the fold to open after the elongate strap is attached to the user, such that a portion of the elongate strap is released as slack to accommodate the limb of the user.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows a perspective view of a wearable device having an ambulatory blood pressure cuff worn by a user, according to aspects of the present disclosure;

FIG. 2A shows an exploded perspective view of the wearable device of FIG. 1, according to aspects of the present disclosure;

FIG. 2B shows a perspective view of the cuff module of the wearable device of FIGS. 1 and 2A, according to aspects of the present disclosure;

FIG. 3A shows a first way of wearing the wearable device of FIG. 1, using a shoulder strap, according to aspects of the present disclosure;

FIG. 3B shows a second way of wearing the wearable device of FIG. 1, using a stretchable fold of an inner sleeve of the wearable device, according to aspects of the present disclosure;

FIG. 4A shows a perspective view of a first example inner sleeve of the wearable device of FIG. 1, according to aspects of the present disclosure;

FIG. 4B shows a perspective view of a second example inner sleeve of the wearable device of FIG. 1, according to aspects of the present disclosure;

FIG. 5A shows a first example sizing mechanism of the wearable device of FIG. 1, according to aspects of the present disclosure;

FIG. 5B shows a second example sizing mechanism of the wearable device of FIG. 1, according to aspects of the present disclosure;

FIG. 6A shows a first fastening mechanism for the cuff of the wearable device of FIG. 1, according to aspects of the present disclosure;

FIG. 6B shows a second fastening mechanism for the cuff of the wearable device of FIG. 1, according to aspects of the present disclosure; and

FIG. 7 shows a block diagram of a wearable device having an ambulatory blood pressure cuff, according to aspects of the present disclosure.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

The present disclosure is susceptible to various modifications and alternative forms, and some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively.

Additionally, directional terminology, such as “top”, “bottom”, “upper”, “lower”, “front”, “back”, “over”, “under”, “above”, “below”, “left”, “right”, etc. is used with reference to the orientation of some of the components in some of the figures described below. Because components in various embodiments can be positioned in a number of different orientations, directional terminology is used for purposes of illustration only and is in no way limiting. The directional terminology is intended to be construed broadly, and therefore should not be interpreted to preclude components being oriented in different ways. These words are intended to relate to the equivalent direction as depicted in a reference illustration; as understood contextually from the object(s) or element(s) being referenced, such as from a commonly used position for the object(s) or element(s); or as otherwise described herein. Further, it is noted that the term “signal” means a waveform (e.g., electrical, optical, magnetic, mechanical or electromagnetic) capable of traveling through a medium such as DC, AC, sinusoidal-wave, triangular-wave, square-wave, vibration, and the like.

Also, as used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

Embodiments of the disclosure relate to devices and systems having ambulatory blood pressure cuffs that can be worn around a limb (e.g., an arm) of a user to, periodically or continuously, measure blood pressure of the user during daily activities of the user. Consistent positioning of a blood pressure cuff is necessary for accurate and consistent measurement of blood pressure during any sort of activity the user may perform during the day. This may be affected by different movements of the user and associated vibrations. The devices and systems described herein address different ways in which a blood pressure cuff can be held at a fixed position on the user's body for blood pressure measurement.

According to embodiments described herein, the device may have an inner sleeve coupled to a cuff module that includes a controller and the blood pressure cuff. The inner sleeve may be easily securable to, and removable from, an arm of a user. The cuff module may include a bladder assembly having one or more bladders that are controllably operated by the controller, and may include a sizing mechanism that enables the blood pressure cuff to maintain a particular tightness around the arm of the user. Advantageously, the blood pressure cuff may be designed to be donned and doffed easily, as well as comfortably worn during daily activities, to continuously measure blood pressure of the user. Further, the sizing mechanisms included on the cuff module may allow the user to repeatedly size the blood pressure cuff to their arm with a maximum allowable extra volume that allows for arm flexure, but without impacting measurement accuracy.

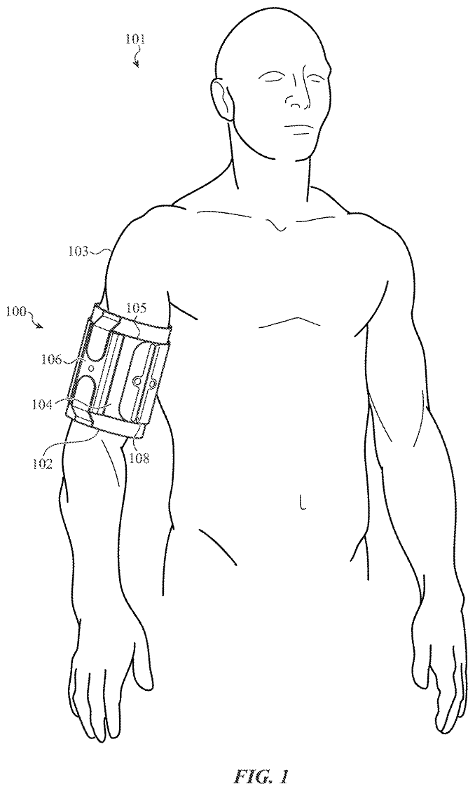

FIG. 1 shows a perspective view of a wearable device 100, worn by a user 101 around an arm 103. The wearable device 100 may include an inner sleeve 102, and a cuff module 104 coupled thereto. The inner sleeve 102 may secure the wearable device 100 to the arm 103 of the user and may be easily separable from the cuff module 104 for cleaning, for example. In some embodiments, the inner sleeve 102 may be transparent in nature to enable accurate optical measurement of the blood pressure of the user 101. In other embodiments, the inner sleeve 102 may be opaque.

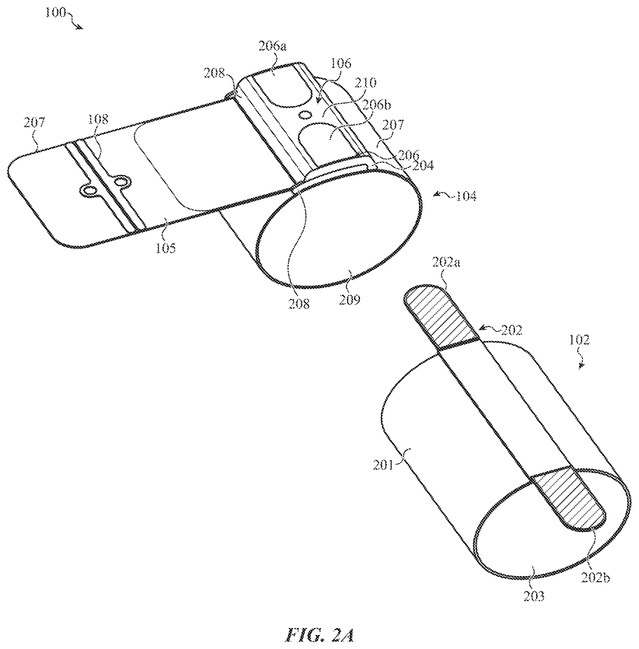

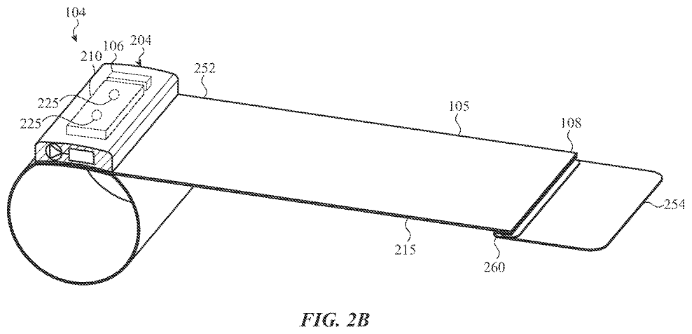

The cuff module 104, as further described in FIGS. 2A-2B, may include a blood pressure cuff 105, a controller 106, and a sizing mechanism 108 that enables the blood pressure cuff to be comfortably worn around the arm 103 of the user 101.

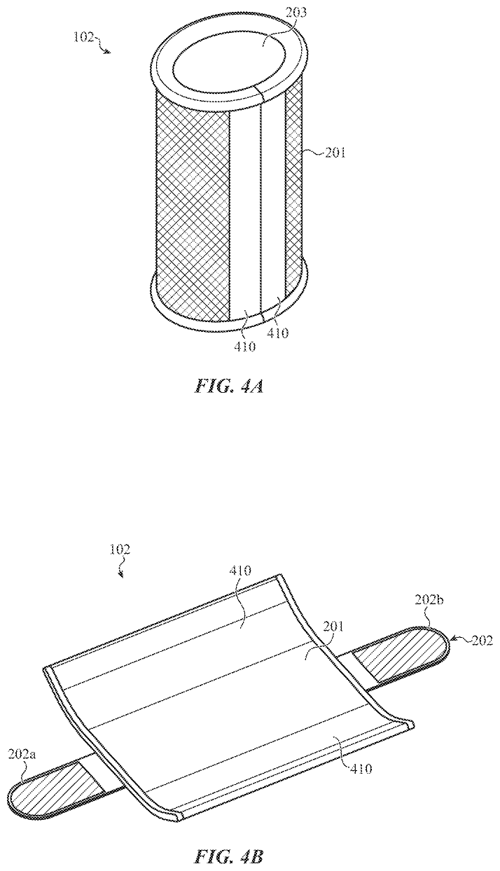

FIG. 2A shows an exploded perspective view of the wearable device 100. The inner sleeve 102 has an outer surface 201 and an inner surface 203. The inner surface 203 may be a breathable and cleanable surface that also provides a level of friction and elasticity, when placed in contact with the skin of the user. In some embodiments, the inner sleeve 102 may be formed from a spacer mesh or another structural fabric having a compression threshold above a maximum expected blood pressure of the user. Additionally or alternatively, in some embodiments, the inner sleeve 102 may have a plurality of stiffeners (as shown in FIG. 4A-4B) embedded between layers of the spacer mesh fabric with perimeter bonding. The plurality of stiffeners may be oriented along a length of the inner sleeve 102 that is perpendicular to a circumference of the inner sleeve 102, which may allow the spacer mesh fabric to stretch despite the static nature of the stiffeners. In some embodiments, the stiffeners can be discretely positioned to avoid a measurement area above an artery of a user's arm. The stiffened nature of the inner sleeve 102 may allow for easy donning and removal without the risk of impacting measurement, while worn, through wrinkling or stiffness irregularities.

A first attachment mechanism 202 may extend from the outer surface 201 of the inner sleeve 102. The first attachment mechanism 202 may be attachable to a second attachment mechanism 206 on the controller 106 of the cuff module 104, and may be used to maintain the blood pressure cuff 105 in position over the inner sleeve 102. In some embodiments, and as shown in FIG. 2A, the first attachment mechanism 202 and the second attachment mechanism 206 may include respective portions of a hook-and-loop fastener. In such embodiments, the first attachment mechanism 202 may include a first tab 202a and a second tab 202b to which a first portion of the hook-and-loop fastener is attached, and the second attachment mechanism 206 may include a first surface 206a and a second surface 206b having a second portion of the hook-and-loop fastener. The first and second portions of the hook-and-loop fastener are complementary portions, and are designed to be attached to one another. The first tab 202a and the second tab 202b may be configured to be folded around the controller 106 and attachably placed over the first surface 206a and the second surface 206b respectively, such that the blood pressure cuff 105 is positioned over the inner sleeve 102. In other embodiments, each of the first attachment mechanism 202 and the second attachment mechanism 206 may each include a respective single tab, with each singular tab including a portion of the hook-and-loop fastener.

In different embodiments, the first attachment mechanism 202 and the second attachment mechanism 206 may include magnetic attachments or mechanical linkages, to attach the cuff module 104 to, and over, the inner sleeve 102.

As shown in FIG. 2A, the cuff module 104 has an outer surface 207 and an inner surface 209. The inner surface 209 may be configured to be disposed over the outer surface 201 of the inner sleeve 102 when the wearable device 100 is worn on the user's arm. The controller 106 and the blood pressure cuff 105 may be coupled to the outer surface 207 of the cuff module 104. The controller 106 may include a housing 204 that is coupled to the outer surface 207 of the cuff module 104 (e.g., via a hinge mechanism or other mechanism 208). As described in further detail below, the sizing mechanism 108 allows a predetermined portion (i.e., length) of the blood pressure cuff 105 to be released as slack to allow flexure of a user's arm.

FIG. 2B shows a perspective view of the cuff module 104 of the wearable device 100 shown in FIGS. 1 and 2A. The cuff module 104 may include a bladder assembly 210, a controller 106, and the blood pressure cuff 105 extending from the bladder assembly 210. The bladder assembly 210 may include one or more bladders and a fluid channel (or channels) that enable fluid to be moved into and out of the bladder assembly 210 (for expansion and contraction of the bladder assembly). One or more sensors 225 may be disposed along the bladder assembly 210 and configured to measure physiological parameters (e.g., blood pressure, heart rate, etc.) of the user, when the blood pressure cuff 105 is wrapped around the arm of the user. The one or more sensors 225 may include, for example, pressure sensors, heart rate monitors, microphones, and the like.

The controller 106 may include a reservoir, a pump, and a power delivery system (e.g., a battery, and conductors to route power delivered by the battery to other components of the controller 106). The controller 106 may pump fluid from the reservoir into the fluid channel through operation of the pump. In some embodiments, the pump may be an ultrasonic pump. The controller 106 may be configured to measure a blood pressure of the user using the one or more sensors of the sensor(s) 225, while controlling expansion and contraction of the bladder(s) in the bladder assembly 210. The power delivery system may deliver power for operating the pump, the sensor(s), and circuitry within the controller 106.

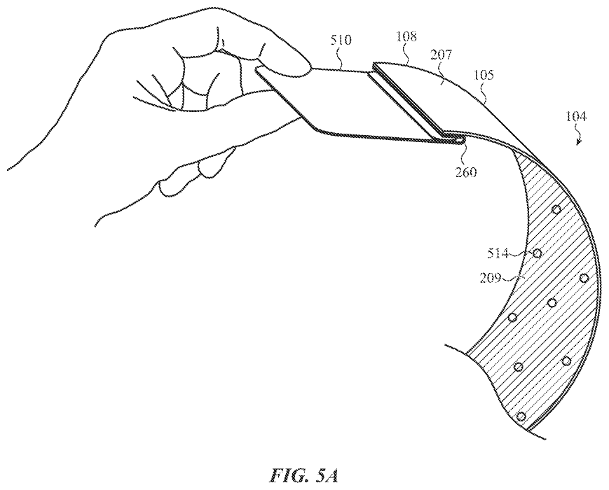

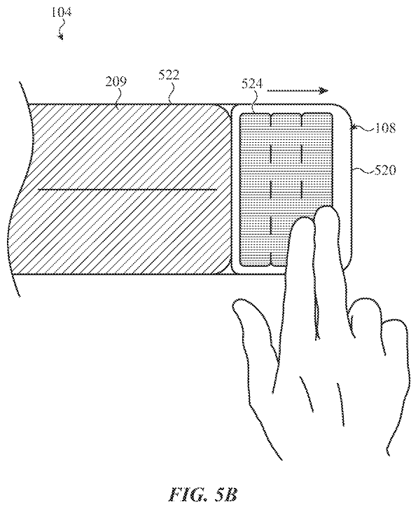

In some embodiments, and as shown in FIG. 2B, the blood pressure cuff 105 may include an elongate strap 215. The elongate strap 215 may have a first end 252 and a second end 254. In some embodiments, the first end 252 may be proximal to the bladder assembly 210 and the second end 254 may be distal from the bladder assembly 210. The second end 254, or another portion of the elongate strap 215, may be configured to attach to the housing 204 or another portion of the elongate strap 215 when the blood pressure cuff 105 is secured to a user. In some embodiments, the elongate strap 215 may be formed from an ultra-high-molecular-weight polyethylene (UHMWPE) laminated between two sheets of polyester such as, but not limited to, Dyneema®. In other embodiments, the elongate strap 215 may include other high tensile-strength yet lightweight and flexible composite fabrics. In some embodiments, an inner surface 209 of the elongate strap 215 may include a plurality of silicon pads (see FIG. 5A) to provide friction for holding the cuff module 104 against the inner sleeve 102.

The elongate strap 215 may have a fold 260 between the first end 252 and the second end 254. The fold 260 may extend along a width of the elongate strap 215. A sizing mechanism 108 allows the fold 260 to open after the elongate strap 215 is wrapped around a user's arm. The sizing mechanism 108 allows the fold 260 to open just enough to release a portion of the elongate strap 215 as slack to accommodate the arm of the user. In some embodiments, the sizing mechanism 108 may include a hook-and-loop fastener, respective complementary portions of which may separate during flexure of a user's arm or expansion of the bladder assembly 210. In other embodiments, the sizing mechanism 108 may include a magnetic attachment, portions of which may separate during flexure of a user's arm or expansion of the bladder assembly 210. In yet other embodiments, the sizing mechanism 108 may include a mechanical linkage (e.g., a linkage having a spring or elastic member that provides slack when a user's arm is flexed).

In some embodiments, the bladder assembly 210 may be expandable and can be operated to transition between an expanded state and a contracted state. The bladder(s) of the bladder assembly 210 may contain a fluid (e.g., air or oil) and be configured to expand and contract in response to an inward or outward flow of the fluid. A fluid channel may extend between the bladder assembly 210 and a reservoir, which reservoir may contain at least a portion of the fluid when the bladder(s) of the bladder assembly 210 are contracted. Alternatively, in the case of a pneumatic bladder system, fluid (e.g., air) may be pulled from and released into an ambient environment and the reservoir may not be provided. The fluid channel may couple the bladder assembly 210 to the reservoir such that fluid can move between the bladder assembly 210 and the reservoir. In some embodiments, the combined fluid volume in the bladder assembly 210, the fluid channel, and the reservoir may be constant. Accordingly, the volume of fluid in the bladder assembly 210 and the reservoir may be inversely related as fluid moves between these two components. For example, fluid can move from the bladder assembly 210, through the fluid channel, and to the reservoir, thereby decreasing the fluid volume in the bladder assembly 210 and increasing the fluid volume in the reservoir.

The bladder assembly 210 may include a length that extends around at least a portion of the circumference of the blood pressure cuff 105 (i.e., around a user's arm) and a thickness that extends from an interior of the blood pressure cuff 105 to an exterior of the blood pressure cuff 105 (transverse to the length). The bladder assembly 210 can be configured such that when it is empty (or nearly empty), the bladder(s) are collapsed. As the bladder assembly 210 is filled with fluid from the reservoir, the volume of the bladder(s) may increase, thereby increasing the thickness of the bladder assembly 210. In some embodiments, the bladder assembly 210 may be formed from a first sheet and a second sheet that are coupled together at defined locations to form the bladder(s). The portions where the first and second sheets are coupled may form sections of the bladder assembly 210 that are impermeable to fluid, and the portions of the first and second sheets that are not coupled may define the bladder(s) and separate/move relative to each other.

The pump may be configured to control a flow of fluid between the reservoir and the bladder assembly 210, thereby switching the bladder assembly 210 between the expanded state and the contracted state. In some embodiments, the pump may include a valve to switchably control the flow of fluid.

In the contracted state, as the user's limb expands, fluid may move from the bladder assembly 210 into the reservoir, thereby allowing the bladder(s) to contract. Also, in the contracted state, the bladder assembly 210 can decrease in size. For example, as a user moves or flexes their arm, fluid can move from the bladder assembly 210 into the reservoir.

In the expanded state, the pump may move fluid into the bladder(s) and then prevent the movement of fluid between the bladder assembly 210 and the reservoir. Since fluid cannot move into or out of the bladder assembly 210, the volume of fluid in the bladder(s) may remain constant. While the bladder assembly 210 is in the expanded state, or as the bladder assembly 210 is expanding toward, or contracting from, the expanded state, one or more sensors of the sensor(s) 225 can be used by the controller 106 to measure a blood pressure of the user. The expanded state of the bladder assembly 210 may make the blood pressure cuff 105 inextensible such that during the blood pressure measurement, the sensors 225 primarily expand toward and compress an arm of a user.

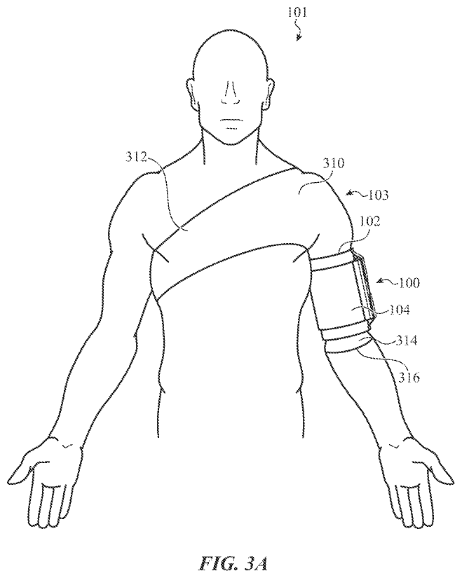

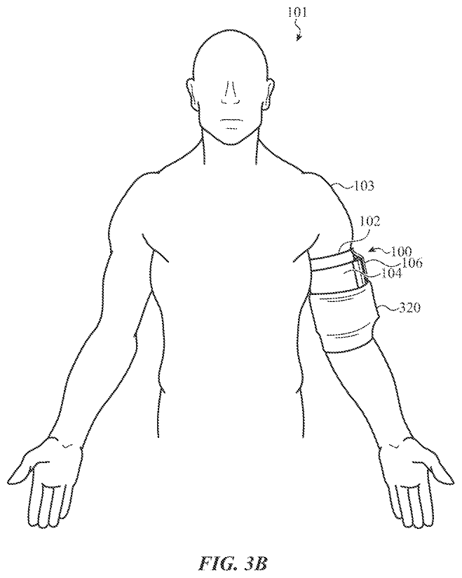

The wearable device 100 may be worn in a way that stabilizes the integrated electronic components in the controller 106 and the blood pressure cuff 105 through a stabilizing component that keeps the wearable device 100 from slipping or rotating from the ideal measurement location on the user's arm. FIG. 3A shows a first way of wearing the wearable device 100, using a shoulder strap 310 attached to the inner sleeve 102. This may improve wearing comfort since the weight of the wearable device 100 is carried by the shoulder of the user, instead of only the arm. The shoulder strap 310 may have a stretchable first portion 312, and an optionally stretchable second portion 314 attached to, or contiguous with, the first portion 312. The first portion 312 may extend over the shoulder and around the torso of the user 101. The second portion 314 may extend around the circumference of an arm 103 of the user 101. The second portion 314 may attach to a lower end 316 of the inner sleeve 102, such that the shoulder strap 310 is configured to double back over at least part of the inner sleeve 102 and the cuff module 104, to aid in securing the wearable device 100 to the user 101.

FIG. 3B shows a second way of wearing the wearable device 100, using a stretchable fold 320 of the inner sleeve 102. After the user 101 dons the inner sleeve 102 around the arm 103, and places the cuff module 104 thereon, the stretchable fold 320 may extend from underneath the cuff module 104 and can double back over at least part of the cuff module 104. In some embodiments, the stretchable fold 320 may include a plurality of silicon pads for holding the cuff module 104 against the inner sleeve 102.

FIG. 4A shows a perspective view of a first example inner sleeve 102 of the wearable device 100, while FIG. 4B shows a perspective view of a second example inner sleeve 102 of the wearable device 100. The inner sleeve 102 may have an outer surface 201 and an inner surface 203. As shown, the outer surface 201 of the inner sleeve 102 may be formed from a spacer mesh or another specialty structural fabric having a compression threshold above a maximum expected blood pressure of the user. A spacer mesh or comparable structural fabric may offer a high level of durability and breathability, with a high compression threshold. In some cases, the spacer mesh may provide a high friction surface that a cuff module 104 may frictionally adhere to, and the spacer mesh may serve as part or all of the first attachment mechanism 202 described with reference to FIG. 2A. In FIG. 4B, the inner sleeve (shown in a collapsed form for storage) is shown to include tabs 202a, 202b that can be fastened to the cuff module 104 by means of a hook-and-loop fastener, magnets, mechanical linkages (e.g., one or more elastic straps) or other types of attachment mechanisms.

As further shown in FIGS. 4A and 4B, a plurality of stiffeners 410 (e.g., rigid or semi-rigid plates or ribs) may be embedded in the inner sleeve 102 between the outer surface 201 and the inner surface 203. The plurality of stiffeners 410 may be oriented along a length of the inner sleeve 102 that is perpendicular to a circumference of the inner sleeve 102.

The wearable device 100 may include a sizing mechanism 108 attached to the cuff module 104, and more particularly to the blood pressure cuff 105. The sizing mechanism 108 may provide slack in the blood pressure cuff 105, which slack allows the user's arm to flex comfortably while wearing the cuff module 104, while maintaining the blood pressure cuff 105 with sufficient tightness around the arm to effectively occlude an artery in the user's arm and measure the user's blood pressure. FIG. 5A shows a first sizing mechanism 108 for wearing the blood pressure cuff 105 in the wearable device 100 around the user's arm. The sizing mechanism 108 (also shown in FIG. 2B) may extend across a width of the elongate strap 215 of the cuff module 104. The sizing mechanism 108 temporarily closes a fold 260 and allows the fold 260 to open. The fold 260 can be one of an S-fold, a Z-fold, and so on. The sizing mechanism 108 may allow the fold 260 to open after the elongate strap 215 is used to attach the cuff module 104 to a user's arm. The fold 260 may open just enough to release a portion of the elongate strap 215 as slack, to better accommodate flexing of a user's arm when a blood pressure measurement is not being taken (but when the wearable device 100 is still being worn). In some embodiments, the sizing mechanism 108 may include a hook-and-loop fastener 510, as shown in FIG. 5A. In other embodiments, the sizing mechanism 108 may include a magnetic attachment. In yet other embodiments, the sizing mechanism 108 may include a mechanical linkage (e.g., one or more springs or elastic bands). In some embodiments, an inner surface 209 of the elongate strap 215 may include a plurality of silicon pads 514, as discussed above.

FIG. 5B shows a second sizing mechanism 108 for the blood pressure cuff 105. The sizing mechanism 108 shown in FIG. 5B includes a portion 520 of the blood pressure cuff 105 that slides over (e.g., is folded over or around) another portion 522 of the blood pressure cuff 105. A mechanical linkage (e.g., one or more springs or elastic bands) allows a relatively small amount of material 524 of the portion 522 to pull out of the portion 520, thereby adding, at most, a predetermined slack to the blood pressure cuff 105.

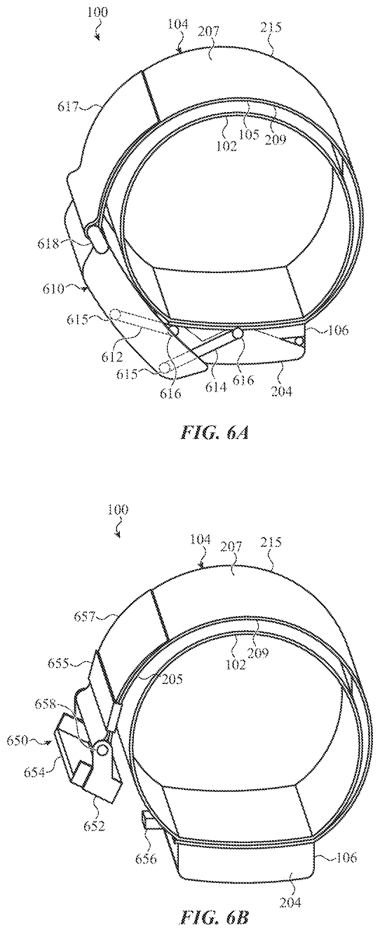

FIG. 6A shows a first fastening mechanism 610 for the blood pressure cuff 105 of the wearable device 100. When fitted around the user's arm, the wearable device 100 is in a closed configuration, where the cuff module 104 is positioned over the inner sleeve 102 adjacent to the inner surface 209 of the blood pressure cuff 105. The first fastening mechanism 610 may include a first mechanical linkage 612, a second mechanical linkage 614, and a rigid bar 618. The first mechanical linkage 612 and the second mechanical linkage 614 may each be rotatably connected between a first set of pivot points 615 on the first fastening mechanism 610 and a second set of pivot points 616 on a housing 204 for the controller 106. As the first mechanical linkage 612 and the second mechanical linkage 614 rotate about the first set of pivot points 615 and the second set of pivot points 616, the first fastening mechanism 610 may move over the housing 204 to secure the blood pressure cuff 105 to a user's arm. At the same time, the elongate strap 215 loops around the rigid bar 618, such that a portion 617 of the elongate strap 215 remains disposed over the outer surface 207 of the blood pressure cuff 105, as needed to accommodate flexure of the user's arm.

FIG. 6B shows a second fastening mechanism 650 for the blood pressure cuff 105 of the wearable device 100. When fitted around the user's arm, the wearable device 100 is in a closed configuration, where the cuff module 104 is positioned over the inner sleeve 102 adjacent to the inner surface 209 of the blood pressure cuff 105. The second fastening mechanism 650 may include a magnetic portion 652 and a rear portion 654 that are held by a rigid bar 658. The magnetic portion 652 may magnetically connect with a corresponding magnetic connector 656 on the housing 204 for the controller 106. As the magnetic portion 652 attaches to the magnetic connector 656 to secure the blood pressure cuff 105 to a user's arm, the elongate strap 215 of the cuff module 104 may loop around the rigid bar 658 and through a clip 655, such that a portion 657 of the elongate strap 215 remains disposed over the outer surface 207 of the blood pressure cuff 105, as needed to accommodate flexure of the user's arm.

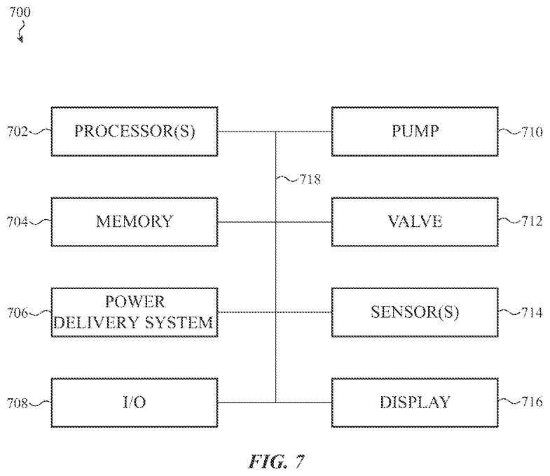

FIG. 7 shows a block diagram of a wearable device 700 having a blood pressure cuff, such as a cuff that is the same as, or substantially similar to, those shown in FIGS. 1-6B. The wearable device 700 may include a processor 702, memory 704, a power delivery system 706, an input/output (I/O) mechanism (or device) 708, a pump 710, one or more valves 712, one or more sensors 714, and an optional display 716.

The processor 702 can control some of all of the operations of the wearable device 700. The processor 702 can communicate, either directly or indirectly, with some or all of the components of the wearable device 700. For example, a system bus or other communication mechanism 718 can provide communication between the processor 702, the memory 704, the power delivery system 706, the input/output (I/O) mechanism 708, the pump 710, one or more valves 712, one or more sensors 714, and the display 716.

The processor 702 can be implemented as any electronic device capable of processing, receiving, or transmitting data or instructions. For example, the processor 702 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements.

It should be noted that the components of the wearable device 700 can be controlled by multiple processors. For example, selected components of the wearable device 700 (e.g., a sensor 714) may be controlled by a first processor and other components of the wearable device 700 (e.g., the display 716) may be controlled by a second processor, where the first and second processors may or may not be in communication with each other.

The memory 704 can store electronic data that can be used by the wearable device 700. For example, the memory 704 can store electrical data or content such as, for example, applications, device settings and user preferences, timing signals, control signals, and data structures or databases. The memory 704 can be configured as any type of memory. By way of example only, the memory 704 can be implemented as random access memory, read-only memory, Flash memory, removable memory, other types of storage elements, or combinations of such devices.

The power delivery system 706 can be implemented with any device capable of providing energy to the wearable device 700. For example, the power delivery system 706 may include one or more batteries or rechargeable batteries. Additionally or alternatively, the power delivery system 706 can include a power connector or power cord that connects the wearable device 700 to another power delivery system, such as a wall outlet.

The 110 mechanism 708 can transmit and/or receive data from a user or another electronic device. An 110 mechanism 708 can include a display, a touch sensing input surface, one or more buttons (e.g., a graphical user interface “home” button), one or more cameras, one or more microphones or speakers, one or more ports, such as a microphone port, and/or a keyboard. Additionally or alternatively, an 110 device or port can transmit electronic signals via a communications network, such as a wireless and/or wired network connection. Examples of wireless and wired network connections include, but are not limited to, cellular, Wi-Fi, Bluetooth, IR, and Ethernet connections.

The wearable device 700 can also include a pump 710 that is used to inflate the bladder. The pump 710 can be an air pump such as an ultrasonic air pump, diaphragm pump, and so on. The pump 710 can be powered by the power delivery system 706 and controlled by the processor 702. The pump 710 may be capable of inflating the bladder to pressures that are above typical human blood pressures such as up to or higher than 180 mm Hg. In some cases, the pump 710 can be configured to apply a linear or smooth pressure ramp within the bladder. The pump 710 can also be configured to provide a linear, or constant, pressure release within the bladder, which may be used to acquire blood pressure measurements during deflation of the cuff.

The wearable device 700 can also include one or more valves 712 that fluidly couple the pump 710 to a bladder. The valves 712 can be passive valves, such as diaphragm valves described herein. In some cases, the valves 712 can be electronically controlled valves such as a solenoid actuated valve. In this regard, the electronically controlled valve can be electrically coupled to the processor 702 such that operation of the valves 712 is controlled by the processor 702.

The wearable device 700 may also include one or more sensors 714 positioned almost anywhere on the wearable device 700. The sensor(s) 714 can be configured to sense one or more types of parameters, such as but not limited to, pressure, sound, light, touch, heat, movement, relative motion, biometric data (e.g., biological parameters), and so on. For example, the sensor(s) 714 may include a pressure sensor, an auditory sensor, a heat sensor, a position sensor, a light or optical sensor, an accelerometer, a pressure transducer, a gyroscope, a magnetometer, a health monitoring sensor, and so on. Additionally, the one or more sensors 714 can utilize any suitable sensing technology, including, but not limited to, capacitive, ultrasonic, resistive, optical, ultrasound, piezoelectric, and thermal sensing technology.

The wearable device 700 may also, optionally, include the display 716. The display 716 may include a liquid-crystal display (LCD), organic light emitting diode (OLED) display, light emitting diode (LED) display, or the like. If the display 716 is an LCD, the display 716 may also include a backlight component that can be controlled to provide variable levels of display brightness. If the display 716 is an OLED or LED type display, the brightness of the display 716 may be controlled by modifying the electrical signals that are provided to display elements. The display 716 may correspond to any of the displays shown or described herein.

The foregoing description, for purposes of explanation, uses specific nomenclature to provide a thorough understanding of the described embodiments. However, it will be apparent to one skilled in the art, after reading this description, that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art, after reading this description, that many modifications and variations are possible in view of the above teachings.

As described above, one aspect of the present technology may be the gathering and use of data available from various sources, including biometric data. The present disclosure contemplates that, in some instances, this gathered data may include personal information data that uniquely identifies or can be used to identify, locate, or contact a specific person. Such personal information data can include, for example, biometric data and data linked thereto (e.g., demographic data, location-based data, telephone numbers, email addresses, home addresses, data or records relating to a user's health or level of fitness (e.g., vital signs measurements, medication information, exercise information), date of birth, or any other identifying or personal information).

The present disclosure recognizes that the use of such personal information data, in the present technology, can be used to the benefit of users. For example, the personal information data can be used to authenticate a user to access their device, or gather performance metrics for the user's interaction with an augmented or virtual world. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide data to targeted content delivery services. In yet another example, users can select to limit the length of time data is maintained or entirely prohibit the development of a baseline profile for the user. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

1. A wearable blood pressure measurement device, comprising:
an inner sleeve wearable on a limb of a user and having a first attachment mechanism; and
a cuff module having a second attachment mechanism, the second attachment mechanism attachable to the first attachment mechanism, the cuff module comprising:
a cuff configured for placement over the inner sleeve;
a bladder assembly having one or more bladders configured to expand and contract in response to a flow of a fluid therethrough; and
a controller configured to measure a blood pressure of the user while controlling expansion and contraction of the bladder assembly.

2. The wearable blood pressure measurement device of claim 1, further comprising a shoulder strap attached to the inner sleeve.

3. The wearable blood pressure measurement device of claim 2, wherein the shoulder strap is attached to a lower end of the inner sleeve, the shoulder strap configured to double back over at least part of the inner sleeve and the cuff module when worn by the user.

4. The wearable blood pressure measurement device of claim 1, wherein the first attachment mechanism and the second attachment mechanism comprise respective portions of a hook-and-loop fastener.

5. The wearable blood pressure measurement device of claim 4, wherein:
the first attachment mechanism comprises a first tab and a second tab to which a first portion of the hook-and-loop fastener is attached;
the second attachment mechanism comprises a second portion of the hook-and-loop fastener; and
the first tab and the second tab are configured to be folded over respective portions of the cuff module, thereby attaching the first portion of the hook-and-loop fastener to the second portion of the hook-and-loop fastener.

6. The wearable blood pressure measurement device of claim 1, wherein the first attachment mechanism comprises at least one tab configured to be folded over a portion of the cuff module and attached to the second attachment mechanism.

7. The wearable blood pressure measurement device of claim 1, wherein the first attachment mechanism and the second attachment mechanism comprise respective portions of a magnetic attachment.

8. The wearable blood pressure measurement device of claim 1, wherein the cuff includes:
an elongate strap having a first end and a second end;
a fold between the first end and the second end in the elongate strap; and
a sizing mechanism that allows the fold to open after the elongate strap is attached to the user, such that a portion of the elongate strap is released as slack to accommodate the limb of the user.

9. A wearable device comprising:
an inner sleeve wearable on a limb of a user;
a measurement module removably coupled to the inner sleeve, the measurement module comprising:
one or more sensors;
a controller in communication with the one or more sensors and configured to measure one or more health parameters of the user using the one or more sensors; and
a power delivery system configured to supply power to the one or more sensors and the controller.

10. The wearable device of claim 9, wherein the inner sleeve comprises:
a fabric; and
a plurality of stiffeners embedded within the fabric and oriented along a length of the fabric, the length perpendicular to a circumference of the inner sleeve.

11. The wearable device of claim 9, wherein the measurement module further comprises a cuff module having one or more bladders configured to expand and contract in response to a flow of a fluid therethrough, and wherein the one or more health parameters include a blood pressure of the user.

12. The wearable device of claim 11, wherein the inner sleeve comprises a spacer mesh having a compression threshold above a maximum expected blood pressure of the user.

13. The wearable device of claim 11, wherein:
the cuff module further comprises a pump configured to expand and contract the one or more bladders; and
the controller is configured to operate the pump to control expansion and contraction of the one or more bladders during measurement of the blood pressure.

14. The wearable device of claim 11, wherein the inner sleeve is configured to double back over at least part of the cuff module when worn by the user.

15. A wearable device for attachment to a limb of a user, comprising:
an elongate strap having a first end and a second end;
a fold in the elongate strap between the first end and the second end; and
a sizing mechanism that allows the fold to open after the elongate strap is attached to the user, such that a portion of the elongate strap is released as slack to accommodate the limb of the user.

16. The wearable device of claim 15, wherein the sizing mechanism includes a hook-and-loop fastener.

17. The wearable device of claim 15, wherein the sizing mechanism includes a magnetic attachment.

18. The wearable device of claim 15, further comprising:
one or more bladders coupled to the elongate strap and configured to expand and contract in response to a flow of a fluid therethrough; and
a controller configured to measure a blood pressure of the user while controlling expansion and contraction of the one or more bladders, thereby measuring the blood pressure of the user.

19. The wearable device of claim 15, wherein the elongate strap is formed from an ultra-high-molecular-weight polyethylene laminated between two sheets of polyester.

20. The wearable device of claim 19, wherein an inner surface of the elongate strap includes a plurality of silicon pads.
